(19) United States
(12) Patent Application Publication
(10) Pub. No.: US 2014/0187537 A1
(43) Pub. Date: Jul. 3, 2014

(54) METHODS OF PREDICTING OUTCOMES OF CHEMOTHERAPY TREATMENTS AND ALTERNATIVE THERAPIES

(71) Applicant: Johann C. Brandes, Atlanta, GA (US)

(72) Inventor: Johann C. Brandes, Atlanta, GA (US)

(73) Assignee: EMORY UNIVERSITY, Atlanta, GA (US)

(21) Appl. No.: 14/146,039

(22) Filed: Jan. 2, 2014

Related U.S. Application Data
(60) Provisional application No. 61/748,464, filed on Jan. 3, 2013.

(51) Int. Cl. C12Q 1/68; A61K 31/337; A61K 31/4184; A61K 31/502; A61K 31/55; A61K 31/496; A61K 31/166

(52) U.S. Cl. CPC C12Q 1/6886; A61K 31/496; A61K 31/337; A61K 31/166; A61K 31/502; A61K 31/55; A61K 31/4184
USPC 514/215; 435/6.11; 514/254.08; 435/287.2; 514/449; 514/248; 514/394

(57) ABSTRACT

This disclosure relates to diagnostic and therapeutic methods and systems related thereto. In certain embodiments, the disclosure contemplates methods of improving chemotherapy treatments by administering chemotherapy agents, analyzing CHFR gene expression, methylation, or both, and determining an appropriate therapeutic strategy. Analysis of data herein revealed that reduced CHFR expression levels was a predictor of improved overall survival of subject with non-small cell lung cancer.

### CROSS REFERENCE TO RELATED APPLICATIONS

This Application claims priority to U.S. Provisional Application No. 61/748,464 filed the 3 of Jan. 2013 hereby incorporated by reference in its entirety.

### GOVERNMENT SUPPORT

This invention was made with government support under Grant No. 1IK2BX001283-01 awarded by the Veterans' Health Administration and under Grant No. 5 P50 CA128613-02 awarded by the National Cancer Institute. The government has certain rights in the invention.

### BACKGROUND

Non-Small Cell Lung Cancers (NSCLCs) are typically carcinomas. Patients with resectable carcinomas typically undergo surgery. However, curing patients with unresectable carcinomas is problematic. The mainstay of therapy in patients with metastatic disease remains systemic chemotherapy. Taxanes, such as paclitaxel and docetaxel play a major role both in first and second line therapy of NSCLC, but overall response rates remain disappointing for some patients. See O'Brien et al., Eur J Cancer, 2003, 39(10): 1416-22. Thus, there is a need to identify improved methods of treating the non-responders.

The identification of biomarkers that are predictive for response serves to allow appropriate targeting of chemotherapy by selecting agents for individual patients with a high likelihood of response. Examples of such predictive markers exist for other therapeutic agents in lung cancer patients where reduced expression of the ERCC1 and RRM1 genes predict sensitivity to platinum compounds and gemcitabine respectively.

Scolnick & Halazonetis report that checkpoint with forkhead and ring finger domains (CHFR) defines a mitotic stress checkpoint that delays entry into metaphase. See Nature 406:430-435, 2000. Yu et al. report CHFR is required for tumor suppression and Aurora A regulation. See Nature genetics 37:401-406, 2005. Herman et al., WO 2009/137094, report identifying a subject that will respond to one or more microtubule-directed therapies comprising: detecting nucleic acid methylation of the checkpoint with forkhead and ring finger domains (CHFR) gene in one or more samples, wherein detecting nucleic acid methylation identifies a subject that will respond to one or more microtubule-directed therapies. Reguart et al., report checkpoint with forkhead and ring finger (CHFR) methylation in serum in erlotinib-treated non-small-cell lung cancer (NSCLC) patients with EGFR mutations. J Clinical Oncology, 2007, ASCO Annual Meeting Proceedings Part I. Vol 25, No. 18S (June 20 Supplement), 2007: 7600. Mariatos et al., report inactivating mutations targeting the CHFR mitotic checkpoint gene in human lung cancer. Cancer Res 63:7185-9, 2003. Koga et al., report aberrant CHFR methylation for clinical response to microtubule inhibitors in gastric cancer. See Journal of gastroenterology 41:133-139, 2006. Banno et al., report inactivation of the CHFR gene in cervical cancer contributes to sensitivity to taxanes. See International journal of Oncology 31:713-720, 2007.

### SUMMARY

This disclosure relates to diagnostic and therapeutic methods and systems related thereto. In certain embodiments, the disclosure contemplates methods of improving chemotherapy treatments by administering chemotherapy agents, analyzing CHFR gene expression, methylation, or both, and determining an appropriate therapeutic strategy. Analysis of data herein revealed that reduced CHFR expression levels was a predictor of improved overall survival of subjects with non-small cell lung cancer.

In certain embodiments, the disclosure relates to methods of predicting the responsiveness of a chemotherapy treatment comprising one, or two, or three or more of the following steps: a) administering a tubulin-targeting agent to a subject diagnosed with cancer; b) measuring a sample for gene expression of checkpoint with forkhead and ring finger domains; c) predicting the responsiveness of the treatment to the tubulin-targeting agent; wherein heightened gene expression of checkpoint with forkhead and ring finger domains indicates an increased risk of the therapy being an ineffective treatment; and d) administering an alternative chemotherapy agent to the subject.

In certain embodiments, the tubulin-targeting agent is a taxane.

In certain embodiments, the taxanes is selected from paclitaxel and docetaxel or combinations thereof.

In certain embodiments, the tubulin-targeting agent is administered in combination with a platinum based agent. In certain embodiments, the platinum based agent is cisplatin.

In certain embodiments, the cancer is non-small cell lung cancer.

In certain embodiments, the subject is a human.

In certain embodiments, measuring gene expression is done by immunohistochemistry or quantifying and or measuring protein, mRNA expression or nuclear DNA transcription by measuring the relative abundance of newly formed transcripts, detecting active transcription sites, measuring the total or nuclear RNA levels, measuring the presence of a transcript, incorporating RNA stem loops sequences into a gene and measuring incorporated the incorporated sequence synthesized RNA by binding a molecule that has a high affinity for sequence-specific interaction with the sequence, directing a fluorescent probe to the site of transcription and visualizing as a fluorescent spot, separating RNA by size such as by electrophoresis and detecting with a hybridization probe complementary to part of target sequence.

In certain embodiments, the sample is a tumor obtained from the subject.

In certain embodiments, the tumor lacks a EGFR mutation, k-ras mutation, has ALK-translocations, or has altered ERCC1 or RRM1 expression levels.

In certain embodiments, the alternative chemotherapy agent is selected from vinorelbine, etoposide, mitomycin C, gemcitabine, irinotecan, pemetrexed, gefitinib, erlotinib, lapatinib, crizotinib, and a vinca alkaloid or combinations thereof. In certain embodiments, the vinca alkaloid is vinblastine, vincristine, vindesine, or vinorelbine.

In certain embodiments, the alternative chemotherapy agent is bevacizumab panitumumab, zalutumumab, nimotuzumab, matuzumab, or cetuximab.

In certain embodiments, the disclosure relates to methods of treating cancer comprising administering an effective amount of a tubulin-targeting agent and a PARP1 inhibitor to subject in need thereof. In certain embodiments, the method is done in combination with administering a platinum based agent.

In certain embodiments, the subject is diagnosed with metastatic non-small cell lung cancer.

In certain embodiments, the tubulin-targeting agent is a taxane. In certain embodiments, the taxane is selected from paclitaxel and docetaxel or combinations thereof.

In certain embodiments, the PARP1 inhibitor is selected from iniparib, olaparib, rucaparib, veliparib, and 3-aminobenzamide, 11-methoxy-2-((4-methylpiperazin-1-yl)methyl)-4,5,6,7-tetrahydro-1H-cyclopenta[a]pyrrolo[3,4-c]carbazole-1,3(2H)-dione including optionally substituted forms, derivatives, or salts, or combinations thereof.

In certain embodiments, the disclosure contemplates a solid surface array comprising probes to CHFR and other biomarkers such as ERCC1 and RRM1.

In certain embodiments, the methods may be implemented by computers, systems, or stored on a computer-readable storage medium as instructions for detecting the CHFR expression.

In some embodiments, the disclosure relates to a system. The system may include a computer having a processor configured to perform the methods of the disclosure. The system may also include or may communicate with a fluorescent camera or other device that can measure light or a change in current of an electrode or system configured to subject a sample to testing device.

In some embodiments, the system may include a computer having a processor configured to perform the methods of the disclosure. In certain embodiments, the method contemplates recording measurements and/or diagnosis and/or second line chemotherapy treatment on a computer readable medium as data. In certain embodiments the disclosure, contemplates reporting measurements or diagnosis to the subject, a medical professional, or a representative thereof. In certain embodiments, the disclosure contemplates transferring recorded data over the internet from a diagnostic lab to a computer in a medical facility.

In some embodiments, the disclosure relates to a system for measuring and recording the gene expression of checkpoint with forkhead and ring finger domains comprising a visual device with a probe that binds to a DNA sequence or RNA sequence, or protein sequence of checkpoint with forkhead and ring finger domains and computer readable memory.

In some embodiments, the method further comprises outputting quantification results. In some embodiments, the method may further comprise recording the detected changes on a computer-readable medium through a visual device such as a camera or video recorder.

### BRIEF DESCRIPTION OF THE FIGURES

FIG. 1 shows data on CHFR promoter methylation that was analyzed by MSP. H226 and H596 lung cancer cell lines were used as controls for unmethylated (U) CHFR, while the Calu-6 cell lines served as control for methylated (M) CHFR. Only 1 sample (LC17) showed CHFR promoter methylation.

FIG. 2 shows nuclear CHFR expression that was analyzed by immunohistochemistry. Shown are samples representative of scores of ‘0’ (a), ‘2’ (b), and ‘4’ (c). Tumors with nuclear expression scores of less than ‘4’ are classified as samples with reduced nuclear CHFR expression.

FIG. 3 shows data on the overall survival analysis by Kaplan-Meier in the original VAMC cohort by (A) response to treatment and (B) CHFR expression levels. C, overall survival in the validation cohort by CHFR expression level.

FIG. 4 shows an example of a system configured to determine CHFR expression with a visual device.

### DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Prior to describing the various embodiments, the following definitions are provided and should be used unless otherwise indicated.

The term “platinum based agent” refers to any of a variety of platinum complexes used as a chemotherapy agent because they interact with DNA. A non-limiting list of examples include: cisplatin, carboplatin, oxaliplatin, satraplatin, picoplatin, nedaplatin, and triplatin.

As used herein, a “taxanes” refers to a 13-[(2R,3S)-3-amino-2-hydroxy-3-phenylpropanoate]-1,7β,10β-trihydroxy-9-oxo-5β,20-epoxytax-11-ene-2α,4,13α-triyl 4-acetate 2-benzoate compound optionally substituted with one or more substituents, or derivatives thereof.

The term “optionally substituted,” as used herein, means that substitution is optional and therefore it is possible for the designated molecule to be unsubstituted. The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ, and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, rodent, monkey or domestic pet.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur or nitrogen atom or replacing an amino group with a hydroxyl group or vice versa. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

As used herein, the term “combination with” when used to describe administration with an additional treatment means that the agent may be administered prior to, together with, or after the additional treatment, or a combination thereof.

The term “sample” as used herein refers to any biological or chemical mixture for use in the method of the invention. The sample can be a biological sample. The biological samples are generally derived from a patient, preferably as a bodily fluid (such as tumor tissue, lymph node, sputum, blood, bone marrow, cerebrospinal fluid, phlegm, saliva, or urine) or cell lysate. The cell lysate can be prepared from a tissue sample (e.g. a tissue sample obtained by biopsy), for example, a tissue sample (e.g. a tissue sample obtained by biopsy), blood, cerebrospinal fluid, phlegm, saliva, urine, or the sample can be cell lysate. In preferred examples, the sample is one or more of blood, blood plasma, serum, cells, a cellular extract, a cellular aspirate, tissues, a tissue sample, or a tissue biopsy. In preferred embodiments, the sample is from esophageal tumor cells, tissue or origin.

Checkpoint with Forkhead and Ringfinger Domains (CHFR)

CHFR is a E3-ubiquitin-ligase and acts as a key regulator for cell cycle entry into mitosis by controlling the activity of the aurora-kinase A and the polo-like kinase 1 and by excluding cyclin B1 from the nucleus. A zinc-finger motif in the C-terminal region of CHFR is a poly-ADP-ribose-binding site. Interactions between CHFR and PARP1 regulate PARP1 levels and seem to be related to CHFR's checkpoint function in response to taxane-induced mitotic stress.

A retrospective cohort study was performed to investigate if either CHFR silencing by DNA methylation or CHFR protein levels could serve as a predictive marker for taxane sensitivity in NSCLC. The significance of nuclear expression of the mitotic checkpoint gene checkpoint with forkhead and ringfinger domains (CHFR) as predictor of response and overall survival with taxane-based firstline chemotherapy in advanced stage NSCLC was investigated.

Data herein indicates a robust and statistically significant correlation between nuclear CHFR expression levels and two important clinical outcomes measures (response and overall survival) after first-line therapy with carboplatin and paclitaxel in NSCLC. These results support the ample preclinical evidence for the role of the CHFR controlled antephase checkpoint in response to microtubular damage: In cells with intact CHFR expression, the antephase checkpoint delays entry into mitosis, prevents nuclear translocation of cyclin D1 and allows cells to repair taxane induced mitotic stress. Cells which are deficient in CHFR expression enter mitosis without delay and undergo mitotic catastrophy, ultimately resulting in apoptosis. These findings have two-fold direct clinical implications: First, reduced CHFR expression levels could be employed to personalize chemotherapy for a large majority of patients with metastatic NSCLC whose tumor lack actionable driver mutations and therefore rely on cytotoxic chemotherapy as primary modality of treatment.

A randomized clinical trial of personalized chemotherapy based on ERCC1 mRNA levels met its primary endpoint of showing superior response rates in the biomarker directed arm compared to the standard-therapy arm. However, no differences in overall survival were observed, presumably because of limited effectiveness of the therapy offered to the ERCC1 high expressing patients or poorer overall prognosis of this subgroup. In order to overcome these limitations particularly in patients with high ERCC1 expression levels and to increase the efficacy of DNA damaging agents such as platinum-compounds, approaches to target DNA repair mechanisms may be used.

The second relevant clinical consequence from this study is the identification of high nuclear CHFR expression as a mechanism of resistance against taxane-based first line therapy in NSCLC. Strategies to target CHFR's function can lead to improvements in response rates to taxane-based first line therapy in metastatic NSCLC. One such approach would be to exploit the interaction between CHFR and PARP1, which is important for CHFR's checkpoint function.

In summary, data herein indicates a robust association between reduced nuclear CHFR expression and response and survival after first-line platinum-taxane combination therapy in NSCLC. These findings will aid in personalize therapy in NSCLC and indicate the use of certain therapies to target CHFR in order to overcome taxane resistance.

The evidence indicates low CHFR expression is linked specifically to taxane—but not platinum-sensitivity: First, overexpression or knockdown of CHFR in vitro is strongly associated with altered response to taxanes, but not DNA damaging agents. Second, in patients with esophageal cancer no association exists between CHFR methylation and response to platinum /non-taxane based combination-therapy. Moreover, in chemonaive patients with resected NSCLC, reduced CHFR expression is associated with a more aggressive phenotype and inferior survival, ruling out the possibility that our findings could be due to an inherently favorable prognosis of patients with reduced CHFR expression.

Epigenetic silencing by promoter DNA methylation does not account for all instances of reduced CHFR expression. The methylation data herein were confirmed by methylation microarray technology which does not require PCR amplification and thus eliminates amplification bias. It is possible that technical differences in methylation specific PCR explain some of the reported differences in methylation frequency in lung cancer cohorts in the literature. For example, a report finding CHFR methylation in circulating cell free DNA in 32% of lung cancer patients utilized an assay that interrogated identical CpG dinucleotides with nearly identical primers as ours, but differed in a higher PCR cycle number and lower annealing temperature, possibly increasing the sensitivity for low levels of CHFR methylation. See Salazar et al., Lung Cancer 2010;72: 84-91.

### Genetic Mutations

In certain embodiments, the disclosure contemplates methods wherein a tumor is evaluated for a EGFR mutation, k-ras mutation, has ALK-translocations, or altered ERCC1 or RRM1 expression levels.

The pathways of EGRF relate to cell growth and survival and depend on stimulation of protein tyrosine kinases Inhibition of EGFR tyrosine kinases in EGFR mutant NSCLC patients leads to better overall survival and response rates. EGFR mutations are most commonly found in females, patients with adenocarcinoma of NSCLC, and those who have never smoked. These tumors are sensitive to EGFR tyrosine kinase inhibitors such as Tarceva and Iressa.

KRAS (V-Ki-ras2 Kirsten rat sarcoma viral oncogene homolog) proteins are Gaunosine Triphosphatases (GTPases). Under the influence of various cell surface receptors such as EGFR they down-regulate Raf/MAP Kinase and PI3 Kinase that help in cell growth and survival. Mutations in KRAS gene active these pathways. Patients with KRAS mutations are typically resistant to EGFR tyrosine kinase inhibitors. KRAS mutation is common in adenocarcinoma of NSCLC and in smokers and is more prevalent in Caucasians than in Asians.

ALK gene typically becomes oncogenic through mutations in the actual gene or by fusion with other genes. The fusion of echinoderm microtubule-associated protein-like 4 gene (EML4) and ALK gene due to an inversion in chromosome 2p often leads to NSCLC. It is often characterized by mucin production, solid growth pattern of signet-ring cells or acinar growth. These tumors are typically sensitive to ALK inhibitors such as Xalkori (crizotinib).

In certain embodiments, this disclosure contemplates methods and systems disclosed herein that additional measure ERCC1 and/or ERCC1 expression. Lord et al., report that low ERCC1 expression correlates with prolonged survival after cisplatin plus gemcitabine chemotherapy in non-small cell lung cancer. Clin Cancer Res 8:2286-91, 2002. Olaussen et al., report DNA repair by ERCC 1 in nonsmall-cell lung cancer and cisplatin-based adjuvant chemotherapy. NEJM 355:983-991, 2006. Zheng et al., report DNA synthesis and repair genes RRM1 and ERCC1 in lung cancer. NEJM 356:800-808, 2007.

### Systems for Measuring Expression

In some embodiments, the determined CHFR expression may be outputted from a visual device. In some embodiments, the outputting may include displaying, printing, storing, and/or transmitting the determined expression. In some embodiments, the determined expression may be transmitted to another system, server and/or storage device for the printing, displaying and/or storing.

The methods of the disclosure are not limited to the steps described herein. The steps may be individually modified or omitted, as well as additional steps may be added.

Unless stated otherwise as apparent from the following discussion, it will be appreciated that terms such as “detecting,” “receiving,” “quantifying,” “mapping,” “generating,” “registering,” “determining,” “obtaining,” “processing,” “computing,” “deriving,” “estimating,” “calculating” “inferring” or the like may refer to the actions and processes of a computer system, or similar electronic computing device, that manipulates and transforms data represented as physical (e.g., electronic) quantities within the computer system's registers and memories into other data similarly represented as physical quantities within the computer system memories or registers or other such information storage, transmission or display devices. Embodiments of the methods described herein may be implemented using computer software. If written in a programming language conforming to a recognized standard, sequences of instructions designed to implement the methods may be compiled for execution on a variety of hardware platforms and for interface to a variety of operating systems. In addition, embodiments are not described with reference to any particular programming language. It will be appreciated that a variety of programming languages may be used to implement embodiments of the disclosure.

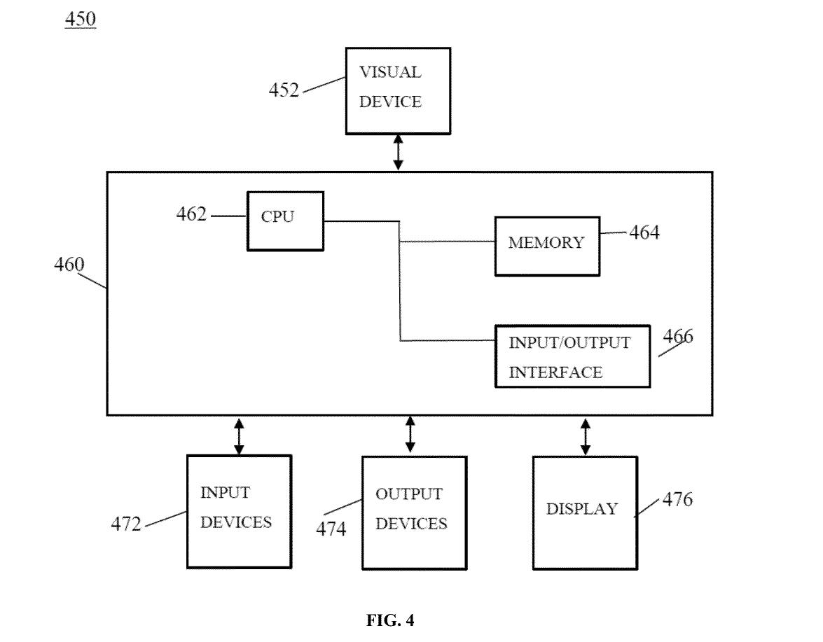

FIG. 4 shows an example of a system 450 that may be used to quantify expression detected by the sensor according to embodiments. The system 450 may include any number of modules that communicate with other through electrical or data connections. In some embodiments, the modules may be connected via a wired network, wireless network, or combination thereof. In some embodiments, the networks may be encrypted. In some embodiments, the wired network may be, but is not limited to, a local area network, such as Ethernet, or wide area network. In some embodiments, the wireless network may be, but is not limited to, any one of a wireless wide area network, a wireless local area network, a Bluetooth network, a radio frequency network, or another similarly functioning wireless network.

Although the modules of the system are shown as being directly connected, the modules may be indirectly connected to one or more of the other modules of the system. In some embodiments, a module may be only directly connected to one or more of the other modules of the system.

It is also to be understood that the system may omit any of the modules illustrated and/or may include additional modules not shown. It is also be understood that more than one module may be part of the system although one of each module is illustrated in the system. It is further to be understood that each of the plurality of modules may be different or may be the same. It is also to be understood that the modules may omit any of the components illustrated and/or may include additional component(s) not shown.

In some embodiments, the modules provided within the system may be time synchronized. In further embodiments, the system may be time synchronized with other systems, such as those systems that may be on the medical and/or research facility network.

The system 450 may optionally include a visual device 452. The visual device 452 may be any visual device configured to capture changes in a shape, light, or fluorescence. For example, the visual device may include but is not limited to a camera and/or a video recorder. In some embodiments, the visual device may be a part of a microscope system. In certain embodiments, the system 450 may communicate with other visual device(s) and/or data storage device.

In some embodiments, the visual device 552 may include a computer system to carry out the image processing. The computer system may further be used to control the operation of the system or a separate system may be included.

The system 450 may include a computing system 460 capable of quantifying the expression. In some embodiments, the computing system 460 may be a separate device. In other embodiments, the computing system 460 may be a part (e.g., stored on the memory) of other modules, for example, the visual device 452, and controlled by its respective CPUs.

The system 460 may be a computing system, such as a workstation, computer, or the like. The system 460 may include one or more processors (CPU) 462. The processor 462 may be one or more of any central processing units, including but not limited to a processor, or a microprocessor. The processor 462 may be coupled directly or indirectly to one or more computer-readable storage medium (e.g., physical memory) 464. The memory 464 may include one or more memory elements, such random access memory (RAM), read only memory (ROM), disk drive, tape drive, etc., or a combinations thereof. The memory 464 may also include a frame buffer for storing image data arrays. The memory 464 may be encoded or embedded with computer-readable instructions, which, when executed by one or more processors 462 cause the system 460 to carry out various functions.

In some embodiments, the system 460 may include an input/output interface 468 configured for receiving information from one or more input devices 472 (e.g., a keyboard, a mouse, joystick, touch activated screen, etc.) and/or conveying information to one or more output devices 474 (e.g., a printing device, a CD writer, a DVD writer, portable flash memory, display 476 etc.). In addition, various other peripheral devices may be connected to the computer platform such as other I/O (input/output) devices.

In some embodiments, the disclosed methods may be implemented using software applications that are stored in a memory and executed by a processor (e.g., CPU) provided on the system. In some embodiments, the disclosed methods may be implanted using software applications that are stored in memories and executed by CPUs distributed across the system. As such, the modules of the system may be a general purpose computer system that becomes a specific purpose computer system when executing the routine of the disclosure. The modules of the system may also include an operating system and micro instruction code. The various processes and functions described herein may either be part of the micro instruction code or part of the application program or routine (or combination thereof) that is executed via the operating system.

It is to be understood that the embodiments of the disclosure may be implemented in various forms of hardware, software, firmware, special purpose processes, or a combination thereof. In one embodiment, the disclosure may be implemented in software as an application program tangible embodied on a computer readable program storage device. The application program may be uploaded to, and executed by, a machine comprising any suitable architecture. The system and/or method of the disclosure may be implemented in the form of a software application running on a computer system, for example, a mainframe, personal computer (PC), handheld computer, server, etc. The software application may be stored on a recording media locally accessible by the computer system and accessible via a hard wired or wireless connection to a network, for example, a local area network, or the Internet.

It is to be further understood that, because some of the constituent system components and method steps depicted in the accompanying figures may be implemented in software, the actual connections between the systems components (or the process steps) may differ depending upon the manner in which the disclosure is programmed. Given the teachings of the disclosure provided herein, one of ordinary skill in the related art will be able to contemplate these and similar implementations or configurations of the disclosure.

### EXAMPLES

### DNA Extraction and Methylation Specific PCR:

DNA was extracted from slides 3 and 4 using the E.Z.N.ATM FFPE DNA extraction kit from Omega Biotek (Norcross, Ga.). DNA content was quantified using an Eppendorf Biophotometer Plus (Eppendorf, Hauppauge, N.J.) with Hellma Tray Cell (Hellma, Mullheim, Germany). Sodium bisulfite modification was performed on 250 ng of DNA using a commercially available kit (EZ DNA methylation kit; Zymo, Irvine, Calif.). This was followed by a 2-step methylation specific PCR for CHFR as described in Brandes et al. Carcinogenesis. 26:1152-1156, 2005.

### Pathologic Review and Immunohistochemistry:

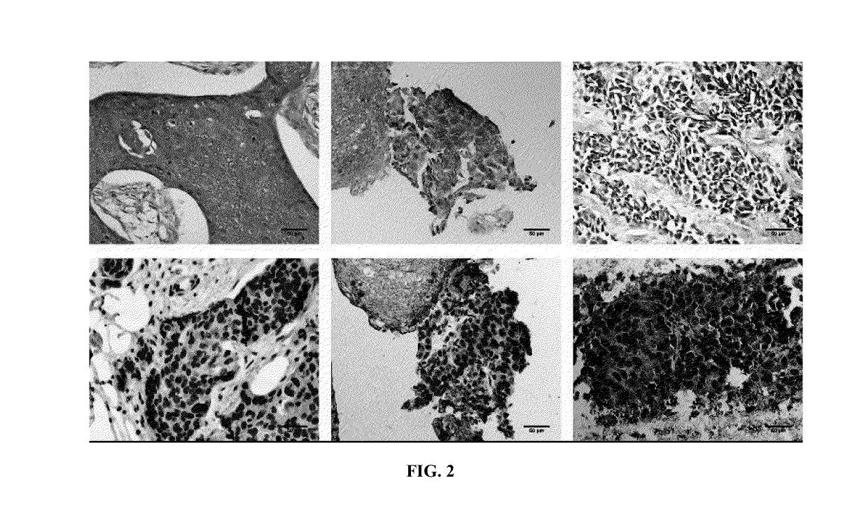

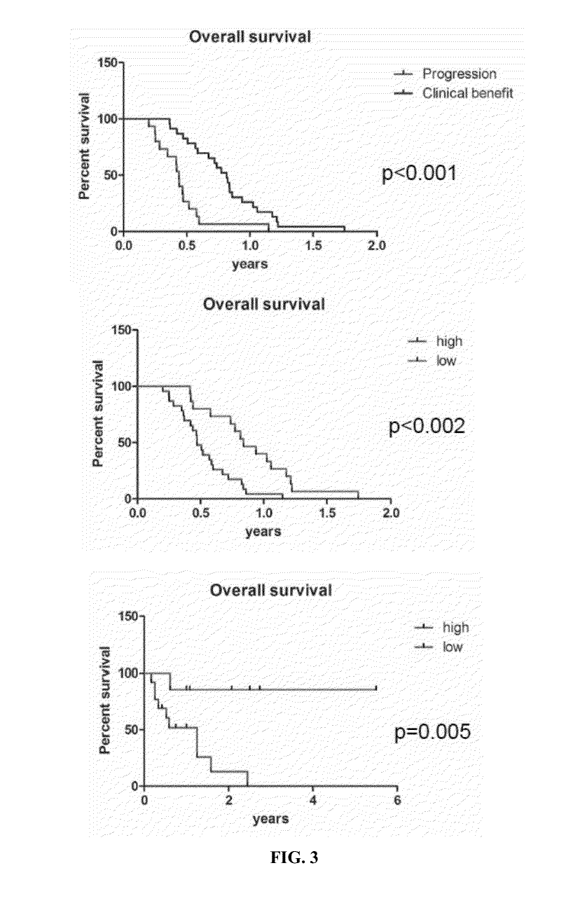

Paraffin blocks were sectioned at 5 um thickness. The first slide of each block was stained with hematoxilyn and eosin (H&E) to confirm original diagnosis and specimen adequacy. The second slide was used for the detection of CHFR protein by immunohistochemistry (IHC). IHC was performed by the Cancer Tissue and Pathology Shared Resource of the Winship Cancer Institute using a monoclonal-rabbit CHFR antibody (Clone, D40H6;Cell Signaling Technology, Danvers, Mass.) in a 1:200 dilution. Staining occurred on a fully automated stainer after standard antigen retrieval steps as previously described. A horseradish-peroxidase labeled secondary anti-rabbit antibody was used in 1:1000 dilution. CHFR staining was scored both for nuclear and cytoplasmic staining based on intensity (0=no staining, 1=weak staining, 2=strong staining) and percentage of cells staining (0<10%; 1: 10-50%; 2>50%)19. See FIG. 2. Scores for intensity and percentage of stained cells were added for a maximum score of ‘4’. Receiver operator characteristics (ROC) were used determine the optimal cut-off value. Scores of ‘4’ were considered “high” expression, while all others were “reduced” expression. See FIG. 3. H&E stained slides and immunohistochemistry were reviewed for accuracy of diagnosis and for scoring by a dedicated lung cancer pathologist (G.S.) who was blinded to the clinical outcomes of the patients.

### Patient Characteristics:

Patients were categorized based on the ECOG performance status into poor (0 and 1) vs. good (2 and 3) status. Response assessment was done by using “Response Evaluation Criteria In Solid Tumors (RECIST 1.1)” criteria. Patients who had received at least 2 cycles of therapy and availability of paraffin-embedded blocks with sufficient tumor tissue to cut at least 4 sections at 5 uM thickness were eligible. After analysis of this original cohort, a validation cohort was assembled of 20 individuals who were either treated either at the Atlanta VAMC between 2011-2012 or Emory University Hospital between 2004-2012.

Between the years from 1999 to 2000 a total of 178 patients received platinum plus taxane-based chemotherapy for stage IV NSCLC at the Atlanta VAMC. Of these, 106 had a biopsy confirmation of disease at our center and had received at least 2 cycles of chemotherapy. Sixty six blocks of paraffin embedded tissue were retrieved from the pathology archives of which 41 had sufficient tumor content of (at least 20% to be used in this study). The vast majority of the patients were males which is representative of the Veterans' Health administration hospital patient population. Patients received carboplatin (CBCDA) and paclitaxel (TAX) with or without bevacizumab (physician decision). Enrolled patients had died at the time of data analysis.

### CHFR Promoter Methylation in NSCLC:

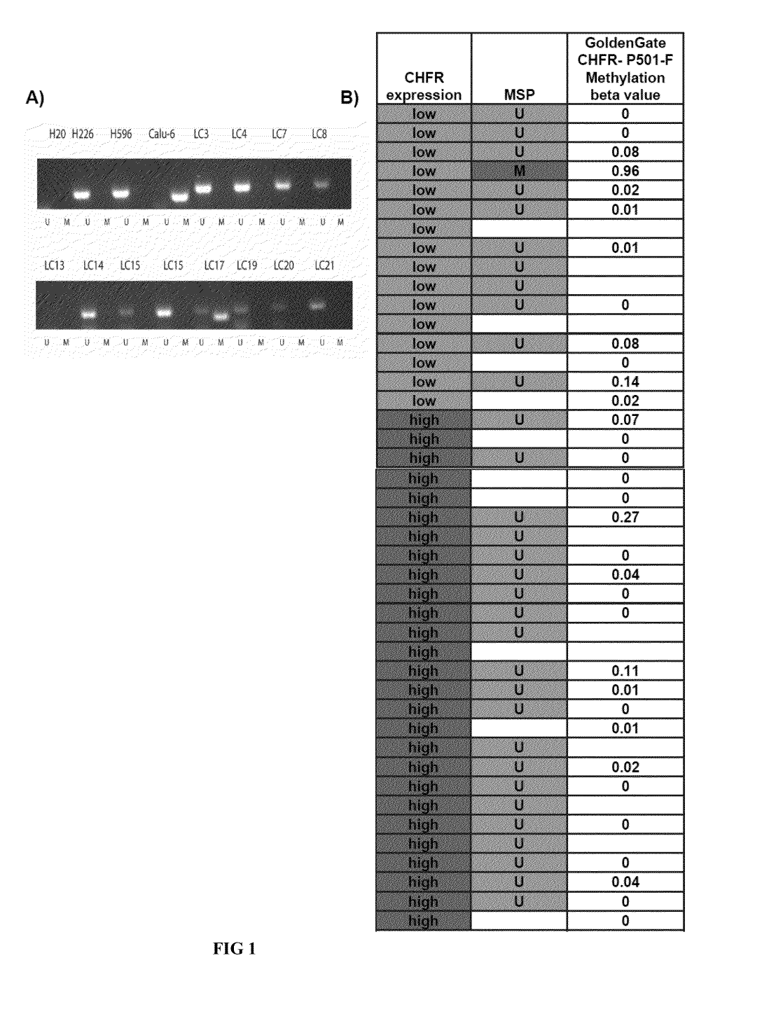

CHFR expression undergoes epigenetic silencing by DNA methylation in 14-18% of NSCLC. Since epigenetic silencing of CHFR expression is in various malignancies and linked to taxane sensitivity, CHFR promoter methylation may serve as a predictive marker for taxane sensitivity also in lung cancer. CHFR promoter methylation was analyzed by qualitative methylation specific PCR (MSP). Amplified bisulfite modified DNA was successfully in 32 samples but DNA methylation was observed in only one sample (3.1%, 95%CI (0.06-15.4%)). See FIG. 1A . These data were compared to CHFR methylation data derived from a methylation microarray (Illumina Goldengate) of the same sample set and found high concordance between MSP and methylation beta-values (p<0.001), indicating that the low frequency of CHFR methylation is unlikely to be explained by technical error. These data were also compared to those derived from a separate cohort of patients with previously resected NSCLC from the Johns Hopkins Hospital. Here, CHFR methylation was observed in 6/65 patients (9%; 95%CI (2.17-16.03%)). Together data from both cohorts indicate that CHFR silencing by promoter methylation is a rare event in NSCLC.

### Nuclear CHFR Expression Predicts Response and Survival:

Whether CHFR protein expression by IHC can be reduced in lung cancer, potentially due to mechanisms other than DNA methylation was investigated. Since CHFR is a checkpoint gene which primarily affects nuclear processes, nuclear staining patterns were focused on for our correlative analysis. Reduced nuclear staining for CHFR was observed in 16 of 41 patient samples. Baseline characteristics for age, gender, race, treatment regimen (CBCDA/TAX vs. CBCDA/TAX/Bevacizumab), histology and date of diagnosis (before or after 2005) were not significantly different between patient groups with high or low CHFR expression. See Table 1A and B.

TABLE 1
A: Patient characteristics by CHFR Expression

| | | | | CHFR | | CHFR | | |
|---|---|---|---|---|---|---|---|---|
| | | Total | | low | | high | | |
| | | N = 41 | | N = 16 | | N = 25 | | p- |
| | | (%) | | (%) | | (%) | | value* |
| Age (years) | Median | 63 | | 65 | | 62 | | 0.13 |
| | Mean | 64 | | 66 | | 62.4 | | |
| | SD | 7.7 | | 8.8 | | 6.7 | | |
| Race | Caucasian | 28 | (68) | 12 | (75) | 16 | (64) | 0.46 |
| | African American | 13 | (32) | 4 | (25) | 9 | (36) | |
| Sex | M | 40 | (98) | 15 | (94) | 25 | (100) | 0.39 |
| | F | 1 | (2) | 1 | (6) | 0 | | |
| Treatment | CBCDA/TAX | 32 | (78) | 12 | (75) | 20 | (80) | 0.72 |
| | Bev/CBCDA/TAX | 9 | (22) | 4 | (25) | 5 | (20) | |
| ECOG PS | 0 | 11 | (27) | 5 | (31) | 6 | (24) | 0.22 |
| | 1 | 15 | (37) | 8 | (50) | 7 | (28) | |
| | 2 | 6 | (15) | 2 | (13) | 4 | (16) | |
| | 3 | 9 | (22) | 1 | (6) | 8 | (32) | |
| Response | CR | 1 | (2) | 1 | (6) | 0 | (0) | 0.034 |
| | PR | 12 | (29) | 4 | (25) | 8 | (32) | |
| | SD | 12 | (29) | 8 | (50) | 4 | (16) | |
| | PD | 16 | (39) | 3 | (19) | 13 | (52) | |
| Histology | Squamous cell | 9 | (22) | 3 | (19) | 6 | (24) | 1.00 |
| | carcinoma (SCC) | | | | | | | |
| | non-SCC | 32 | (78) | 13 | (81) | 19 | (76) | |
| Number of | 0 | 26 | (63) | 9 | (56) | 17 | (68) | 0.22 |
| lines of | 1 | 10 | (24) | 3 | (19) | 7 | (28) | |
| additional | 2 | 4 | (10) | 3 | (19) | 1 | (4) | |
| therapy | 3 | 1 | (2) | 1 | (6) | 0 | (0) | |
| Age-category | >=65 | 17 | (41) | 8 | (50) | 9 | (36) | 0.38 |
| | <65 | 24 | (59) | 8 | (50) | 16 | (64) | |
| ECOG PS- | good | 26 | (63) | 13 | (81) | 13 | (52) | 0.06 |
| category | poor | 15 | (37) | 3 | (19) | 12 | (48) | |
| Response- | Clinical benefit | 25 | (61) | 13 | (81) | 12 | (48) | 0.033 |
| category | Progression | 16 | (39) | 3 | (19) | 13 | (52) | |
| Time of | before 2005 | 14 | (34) | 5 | (31) | 9 | (36) | 0.75 |
| diagnosis | 2005 and later | 27 | (66) | 11 | (69) | 16 | (64) | |
| 2 or more lines | no | 26 | (63) | 9 | (56) | 17 | (68) | 0.446 |
| of treatment | yes | 15 | (37) | 7 | (44) | 8 | (32) | |

B: Patient characteristics of the validation set

| | | | | CHFR | | CHFR | | |
|---|---|---|---|---|---|---|---|---|
| | | total | | low | | high | | p- |
| | | 20 | | 7 (%) | | 13 (%) | | value* |
| Age | Mean | | | 67.4 | | 61.5 | | 0.3 |
| | Median | | | 69 | | 66 | | |
| Race | C | 12 | (60) | 4 | (58) | 8 | (62) | 1 |
| | AA | 6 | (30) | 2 | (28) | 4 | (31) | |
| | unknown | 2 | (10) | 1 | (14) | 1 | (7) | |
| Sex | M | 16 | (80) | 5 | (71.4) | 11 | (84.6) | 0.48 |
| | F | 4 | (20) | 2 | (28.6) | 2 | (15.4) | |
| Treatment | CDDP/TAX | 17 | (85) | 5 | (71) | 12 | (70) | 0.33 |
| | CDDP/TAX/Avastin | 3 | (15) | 2 | (29) | 1 | (30) | |
| ECOG PS | 0 | 4 | (20) | 2 | (29) | 2 | (15) | 0.84 |
| | 1 | 6 | (30) | 2 | (29) | 4 | (31) | |
| | 2 | 6 | (30) | 2 | (29) | 4 | (31) | |
| | unknown | 4 | (20) | 1 | (15) | 3 | (23) | |
| Histology | SCC | 1 | (5) | 0 | (0) | 1 | (7.7) | 0.34 |
| | non-SCC | 19 | (95) | 7 | (100) | 12 | (92.3) | |
| Clinical | yes | 11 | (55) | 7 | (100) | 4 | (31) | 0.03 |
| benefit | non | 8 | (40) | 0 | (0) | 8 | (62) | |
| | Unknown | 1 | (5) | 0 | (0) | 1 | (7) | |
| Time of | Before 2005 | 2 | (10) | 1 | (14.3) | 1 | (7.7) | 0.64 |
| diagnosis | After 2005 | 18 | (80) | 6 | (85.7) | 12 | (92.3) | |
| 2 or more | yes | 3 | (15) | 2 | (29) | 1 | (8) | |
| lines of | no | 17 | (85) | 5 | (71) | 12 | (92) | 0.22 |
| therapy | | | | | | | | |

The year 2005 was chosen as cutoff because it was around this time when second line therapy with pemetrexed and antiangiogenic therapy with bevacizumab emerged, resulting in improved overall survival rates. The subgroup of patients with low nuclear CHFR expression had trends towards having a better ECOG performance status (0 and 1 vs. 2 and 3), p=0.058 and towards a higher rate of subsequent therapies (44% vs. 32%, p=0.446).

Reduced nuclear CHFR expression in 16 patients (37%) showed a statistically significant association with response to therapy as determined at first restaging (19% progression vs. 52% progression, p=0.033) . Kaplan-Meier analysis and univariate Cox models showed a strong correlation between clinical benefit at first restaging and overall survival (Median survival 9.4 months vs. 5.1 months, HR 0.28 (95%CI 0.14-0.56), p<0.001). See FIG. 3. Low nuclear CHFR expression was also strongly predictive of improved survival (median survival 9.9 months vs. 5.7 months, HR 0.32 (95%CI 0.16-0.67, p=0.002). To account for potential confounders of these results, particularly in light of the slightly uneven distribution of patients with good vs. poor performance status, a multivariate Cox proportional hazard analysis was performed. After multivariate adjustment, reduced nuclear CHFR expression emerged as an even more powerful predictor of survival (HR 0.24 (95% CI 0.1-0.58, p=0.002). See Table 2. Second line of treatment was the only other covariate which was significantly associated with survival.

These results were validated in our second cohort: Low CHFR expression was associated with a higher likelihood of achieving a clinical benefit (100% vs. 31%, p=0.03) and improved overall survival (median survival CHFR high: 1.25 years vs. CHFR low—not yet reached HR 0.09 (95% CI 0.004-0.5), p=0.006). This association was confirmed after multivariate adjustment (HR 0.11(95%CI 0.01-0.88), p =0.038) (Table2).

TABLE 2
Univariate and multivariate adjusted hazard ratio for overall survival

| | | | Multivariate | |
|---|---|---|---|---|
| | | | adjusted | |
| | Crude HR | | HR* (95% | |
| | (95% CI) | p-value | CI) | p-value |
| | Atlanta VAMC cohort | | | |
| CHFR nuclear stain high vs. | 3.09 | 0.001 | 4.18 | 0.002 |
| low | (1.5-6.4) | | (1.71-10.18) | |
| Age (<65 vs. >=65) | 1.60 | 0.16 | 1.96 | 0.125 |
| | (0.82-3.12) | | (0.83-4.65) | |
| Gender (Male vs. female) | 1.5 | 0.69 | † | |
| | (0.7-25.7) | | | |
| Race (AA vs. C) | 1.07 | 0.85 | † | |
| | (0.54-2.09) | | | |
| Treatment | 1.405 | 0.38 | * | |
| (CBCDA/TAX vs. | (0.65-3.01) | | | |
| CBCDA/TAX/Bevacizumab | | | | |
| Histology (SCC vs non- | 1.6 | 0.25 | † | |
| SCC) | (0.73-3.36) | | | |
| Performance status (good | 0.57 | 0.09 | † | |
| vs. poor) | (0.29-1.11) | | | |
| Time of diagnosis (before | 0.95 | 0.87 | * | |
| 2005 vs. later) | (0.48-1.86) | | | |
| Second line treatment | 3.254 | <0.001 | 6.32 | <0.001 |
| (no vs. yes) | (1.59-6.66) | | (2.58-15.51) | |
| | Validation cohort | | | |
| CHFR nuclear stain high vs. | 10.9 | 0.006 | 9.15 | 0.038 |
| low | (1.36-88) | | (1.14-104.84) | |
| Age (<65 vs. >=65) | 1.4 | 0.58 | 1.37 | 0.67 |
| | (0.40-4.76) | | (0.32-5.88) | |
| Gender (Male vs. female) | 2.15 | 0.45 | † | |
| | (0.4-39.7) | | | |
| Race (AA vs. C) | 1.27 | 0.70 | † | |
| | (0.36-4.27) | | | |
| Treatment | 2.67 | 0.31 | * | |
| (CBCDA/TAX vs. | (0.50-4.92) | | | |
| CBCDA/TAX/Bevacizumab | | | | |
| Histology (SCC vs non- | 4.21 | 0.16 | † | |
| SCC) | (0.22-28.5) | | | |
| Performance status (good | 0.51 | 0.31 | † | |
| vs. poor) | (0.12-4.58) | | | |
| Time of diagnosis (before | 0.28 | 0.10 | * | |
| 2005 vs. later) | (0.06-1.96) | | | |
| Second line treatment | 4.16 | 0.15 | 2.21 | 0.49 |
| (no vs. yes) | (0.75-78) | | (0.24-20.5) | |

What is claimed:

1. A method of predicting the responsiveness of a chemotherapy treatment comprising
a) administering a tubulin-targeting agent to a subject diagnosed with cancer;
b) measuring a sample for gene expression of checkpoint with forkhead and ring finger domains;
c) predicting the responsiveness of the treatment to the tubulin-targeting agent;
wherein heightened gene expression of checkpoint with forkhead and ring finger domains indicates an increased risk of the therapy being an ineffective treatment; and
d) administering an alternative chemotherapy agent to the subject.

2. The method of claim 1, wherein the tubulin-targeting agent is a taxane.

3. The method of claim 2, wherein the taxane is selected from paclitaxel and docetaxel or combinations thereof.

4. The method of claims 1-3, wherein the tubulin-targeting agent is administered in combination with a platinum based agent.

5. The method of claim 4, wherein the platinum based agent is cisplatin.

6. The method of claims 1-5, wherein the cancer is non-small cell lung cancer.

7. The method of claims 1-6, wherein the subject is a human.

8. The method of claims 1-7, wherein the sample is a tumor obtained from the subject.

9. The method of claim 8, wherein the tumor lacks a EGFR mutation, k-ras mutation, ALK-translocations, or altered ERCC1 or RRM1 expression levels.

10. The method of claims 1-9, wherein the alternative chemotherapy agent is selected from vinorelbine, etoposide, mitomycin C, gemcitabine, irinotecan, pemetrexed, gefitinib, erlotinib, lapatinib, crizotinib, and a vinca alkaloid or combinations thereof.

11. The method of claim 10, wherein the vinca alkaloid is vinblastine, vincristine, vindesine, or vinorelbine.

12. The method of claims 1-9, the wherein the alternative chemotherapy agent is bevacizumab panitumumab, zalutumumab, nimotuzumab, matuzumab, or cetuximab.

13. A method of treating cancer comprising administering an effective amount of a tubulin-targeting agent and a PARP1 inhibitor to subject in need thereof.

14. The method of claim 13, wherein the subject is diagnosed with metastatic non-small cell lung cancer.

15. The method of claim 13, wherein the tubulin-targeting agent is a taxanes.

16. The method of claim 15, wherein the taxanes is selected from paclitaxel and docetaxel or combinations thereof.

17. The method of claim 13-16, wherein the PARP1 inhibitor is selected from iniparib, olaparib, rucaparib, veliparib, and 3-aminobenzamide, 11-methoxy-2-((4-methylpiperazin-1-yl)methyl)-4,5,6,7-tetrahydro-1H-cyclopenta[a]pyrrolo[3,4-c]carbazole-1,3(2H)-dione, or substituted forms, or salts, or combinations thereof.

18. A system for measuring and recording the gene expression of checkpoint with forkhead and ring finger domains comprising a visual device with a probe that binds to a DNA sequence or RNA sequence, or protein sequence of checkpoint with forkhead and ring finger domains and computer readable memory.
